(19) United States
(12) Patent Application Publication
(10) Pub. No.: US 2005/0037437 A1
(43) Pub. Date: Feb. 17, 2005

(54) Identification of antigen by xenogenic, allogenic or autologous antibody-mediated precipitation

(76) Inventor: Reinhard Zeidler, Olching (DE)

Correspondence Address: FISH & RICHARDSON PC, 225 FRANKLIN ST, BOSTON, MA, 02110, US

(21) Appl. No.: 10/489,516

(22) PCT Filed: Sep. 13, 2002
(86) PCT No.: PCT/EP02/10318

(30) Foreign Application Priority Data
Sep. 14, 2001 (DE) 101453841
Oct. 16, 2001 (DE) 101510519

(51) Int. Cl. G01N 33/53

(52) U.S. Cl. 435/7.2

(57) ABSTRACT

The invention relates to a method for identifying antigens that are associated with diseases, in which a humoral immune response occurs and specific antibodies are formed. The method is based on the autologous, allogenic or xenogenic antibody-mediated precipitation of antigens from cell lysates or bacterial, parasitic and/or viral preparations comprising autologous, allogenic or xenogenic serums, ascites or pleural fluids.

The present invention relates to a method (herein termed AMIDA, for antibody-mediated Identification of antigens) for the identification of antigens, associated with a disease characterized by a humoral immune response and the generation of specific antibodies. This method is based upon an autologous, allogeneic, or xenogeneic antibody-mediated precipitation of antigens from cell lysates or preparations of bacteria, parasites or viruses with autologous, allogeneic, or xenogeneic sera, ascites, or pleural fluids. The induction of an immune response, which is accompanied by the production of antibodies, is thus the only prerequisite for the application of AMIDA. Above all, this method is suitable for the identification of tumor antigens, but also of antigens associated with autoimmune diseases and those associated with infections by bacteria, viruses or parasites.

‘Antigens’ (as used herein) are structures that provoke the generation of antibodies, since they are ‘foreign’ to the immune system. Specific antigens are a prerequisite for the diagnosis and immunotherapy of tumor patients and patients suffering from e.g. an autoimmune disease or from chronic infections. Such antigens, being more or less specific for a given disease, allow for the proof and the targeting of tumor cells, of cells that are targets of an autoimmune reaction, and of infected cells and infectious organisms. Here, a method for the isolation and identification of antigens, that are targets of a humoral immune response, is being described.

Targeting of cells in vivo and in vitro with immunological tools like T cells and antibodies strongly depends upon the knowledge about specific proteins on target cells. Consequently, many groups have put in much effort to identify and characterize such antigens, like tumor-specific antigens for many years. This has preferably been done and still is being done by analyzing the repertoire of specific T cells (cellular immune response). In contrast, the importance and the quality of the humoral immune response is not adequately understood. Characterization of the immune response is mainly based upon techniques that allow for the molecular cloning of such tumor antigens, which are targets of a humor immune response and induce specific antibodies.

The existence of tumor-specific antibodies against known antigens like p53 and HER-2/neu has been extensively described in tumor patients. The identification of new antigens by the use of autologous or allogeneic antibodies has mainly become possible with the SEREX-technology (Serological analysis of recombinant cDNA expression libraries; published in: “Human neoplasms elicit multiple specific immune responses in the autologous host” Ugur Sahin et al, PNAS Vol 92 pp 11810-11813 December 1995). Using SEREX, Pfreundschuh and colleagues have demonstrated that tumors induce the activation of CD4+ T cells, leading to the activation of antigen-specific B cells and eventually the production of antibodies. Tumor-specific antibodies are suitable tools for the isolation of specific antigens. Known antigens like MAGE-1 and Tyrosinase have been cloned with SEREX. In addition, totally new antigens have been identified, the most popular being NY-ESO-1, which is found in many kinds of cancer like melanoma and cancer of the breast and the urinary bladder, in prostate cancer and in hepatocellular cancer. In healthy tissues, expression of NY-ESO-1 is restricted to testis and ovary.

The SEREX technology comprises the following steps:
1. Generation of a cDNA library from a tumor biopsy and cloning into a γ-ZAP vector;
2. Infection of suitable E. coli and expression of the cloned cDNAs in these bacteria;
3. Transferring these recombinant E. coli onto a nitrocellulose membrane and screening of the colonies with autologous sera;
4. Detection of bound antibodies with an anti-IgG-antibody;
5. Amplification of positive colonies, isolation of the containing plasmids;
6. Sequencing and identification of the containing cDNA.

The major drawback of SEREX is that the method is very time-consuming and complicated with respect to its technical application. It can easily take up to six month to analyze a single tumor biopsy. Another problem with SEREX is the heterologous expression of cDNAs isolated from a tumor biopsy in E. coli: since posttranslational modifications like glycosylation (which may be essential for the recognition of the antigen by specific antibodies) do not take place or take place in a modified fashion, recognition of antigens is not secured. In addition, SEREX is based upon a series of steps like reverse transcription, cloning, and transcription, any one being error-prone. In summary, SEREX is a technology with a certain potential, which is time-consuming and error-prone.

It is thus the aim of the present invention, to provide a better and more simple method for the isolation and identification of antigens, which can be performed directly, easily, and within a short period of time.

This task will be solved as indicated in claim 1. Preferred applications of the invention are defined in the subclaims.

The present invention provides an efficient method for the isolation and identification of antigens and is based upon a species-specific, xenogeneic, or autologous system that makes use of antibodies present in sera, ascites, and/or pleural fluids. The advantages of the present invention are the following: the system does not depend on heterologous expression of cDNAs in bacteria or eukaryotic recipient cells, but rather allows for direct screening in a native context, i.e. the tumor cells or diseased tissues themselves, as well as in bacteria, viruses, or parasites themselves. This point is of special importance since an ectopic expression of antigens in non-related eukaryotic recipient cells or bacteria is not similar to the expression within the donor cells (e.g. cells from a tumor biopsy): post-translational modifications like glycosylation differ depending on the expression system or does not occur all (in bacteria). However, glycosylation can be essential for antigens to be recognized by antibodies.

In addition, the present invention can be established as a high-throughput and semi-automatic technology, allowing for the investigation of more than one patient in parallel. The method is fast, and it is realistic to isolate and completely identify a tumor-associated protein within a period of only 1-2 weeks.

The present invention is suitable for the investigation of different antigens: tumor antigens, above all, but also antigens associated with auto-immune diseases and antigens associated with infections by bacteria, parasites, or viruses.

In addition to the identification of tumor-associated antigens, the technique can be used for the investigation of antigens associated with auto-immune diseases. As holds true for tumors, most antigens that are targets of autoimmune reactions, are not known. In some cases, a recognition of a certain antigen by specific T cells could be demonstrated. However, the role of B cells and antibodies in autoimmune diseases is largely unknown. Hence, the present invention may contribute to a better insight into and understanding of some of these diseases.

It is also possible to use the present invention for chronic diseases that are caused by a known pathogenic microorganism. Tissue from a patients or an animal model based on different mouse strains can be used equally. Mouse strains being either resistant against or susceptible to a certain bacterial infection can be used in order to characterize the humoral immune response responsible for the protection of resistant mice. The application of human tissue allows for the validation of the results/antigens obtained from the animal model and thus for the identification of therapeutically interesting antigens. Also, infections with different parasites (e.g. plasmodia) or viruses (e.g. members of the family of the herpes viruses) induce immune reactions and the generation of specific antibodies. Thus, the present invention can also be used for these diseases in order to identify new antigens and to improve therapeutic options.

Once isolated and characterized, tumor antigens, auto-antigens, and immunogenic proteins derived from bacteria, parasites, or viruses can be used in multiple ways: (i) dendritic cells or B cells can be loaded with these proteins and can be used for the activation of specific T cells, (ii) monoclonal and bispecific antibodies can be produced that may be of a great value for the diagnosis of diseases and the therapy of patients. Thus, the present invention represents a novel and promising tool for investigating the humoral immune response in patients with different diseases and thus contributes significantly to the development of new therapies.

The present invention comprises the following steps:

First, a biopsy derived from the tissue of interest is taken from a donor and a protein lysate is generated from this biopsy. Alternatively, lysates are prepared from bacteria, parasites, or viruses. In order to obtain a specific binding of antigens to antibodies, this protein lysate is then incubated with allogeneic, xenogeneic and/or autologous sera, ascites or pleural fluids, containing antibodies that have been generated in the course of a humoral immune response and that recognize these antigens. Thereafter, antibody-antigen-complexes are separated and the antigens that are recognized by specific antibodies are identified with suitable methods.

As used herein, ‘antigen’ refers to any substance or structure, which can specifically be recognized by antibodies, especially a substance or structure that provokes an immune response in vertebrate. In the context of the present invention, ‘antigen’ means a tumor antigen or a target antigen associated with humoral immune responses in the course of autoimmune diseases or infections (by bacteria, viruses or parasites).

As used herein, ‘autologous’ means that the donor of a biopsy or tissue and the donor of the antibody-containing serum is identical.

As used herein, ‘allogeneic’ means that the donor of a biopsy or tissue and the donor of the antibody-containing serum are not identical but rather genetically different but belong to the same species.

As used herein, ‘xenogeneic’ means that the donor of a biopsy or tissue and the donor of the antibody-containing serum are not identical but rather genetically different and do not belong to the same species.

Although the present invention is preferably performed in an autologous system, allogeneic or xenogeneic sera, ascites and/or pleural fluids containing antibodies can be used as well. Many species-specific antigens, especially tumor antigens, can be identified this way, without being absolutely dependent on autologous sera. A xenogeneic application form of the present invention is e.g. the incubation of a protein lysate derived from bacteria or viruses with sera from infected organisms.

As used herein, ‘serum’ refers to the clear fluid of blood, containing antibodies and other proteins.

In the context of the present invention, ‘donor’ means an individual who provides antigens to be identified by applying the present invention with xenogeneic, allogeneic or autologous sera, ascites or pleural fluids containing antibodies. Preferably, donors are either mammals or human beings, but may also be infectious organisms, microorganisms or cell lines.

‘Autoimmune diseases’ are autoaggressive diseases that provoke immune response characterized by the generation of T cells and antibodies, which recognize self-antigens. Examples for autoimmune diseases are Hashimoto-tyreoditis, pernicious anemia, chronic gastritis, Addison-disease, systemic lupus erythematodes, multiple sclerosis, and many others. Here, as well as in the infections with bacteria, parasites and viruses (as mentioned above), the present invention is applied whenever the target antigens of the humoral immune response are not known or not sufficiently investigated.

In an embodiment of the invention described herein, antibodies directed against immunoglobulins of the donor (e.g. subclass IgG) are added during the process of incubation of the protein lysate with autologous, allogeneic or xenogeneic sera, ascites or pleural fluids containing antibodies. These additional antibodies are coupled to a matrix and thus serve as a ‘bridge’ between the matrix (e.g. sepharose) and antibodies present in e.g. serum. This way (and if desired), a certain subclass of serum antibodies can be bound specifically to the matrix, introducing additional specificity into this technology.

As used herein, protein lysates are generated by lysing cells with detergents (e.g. triton-x-100, NP40), leading to complete disintegration of the cell membrane. Then, the particulate fraction is precipitated while the great majority of the proteins remains in solution.

Alternatively, the protein lysate is fractionated prior to incubation, dividing the lysate into a membrane and a cytoplasmatic fraction. Both fractions can then be used separately, allowing for an additional specification of the isolated antigens and for a simplified technical execution of the present invention, since a smaller number of proteins needs to be separated. Protein fractions can also be generated to enrich certain cellular compartments. Alternatively, proteins can be fractioned according to their molecular weight with size exclusion columns.

Preferably, antibodies used for the present invention are coupled to a matrix. This holds true for antibodies being used for incubation with the protein lysate and present in autologous/allogeneic/xenogeneic sera, ascites or pleural fluids, as well as for antibodies that are possibly added, being directed against immunoglobulins of the donor. Binding to a matrix allows subsequent precipitation of antigen-antibody-complexes via centrifugation. E.g. sepharose-beads coated with antibodies sediment due to their specific weight. Matrices consist of either sepharose, sepharose protein A, sepharose protein G, agarose protein A, or agarose protein G.

Alternatively, the protein lysate is pre-incubated with the matrix (=pre-clearing), allowing the removal of proteins, which bind to the matrix unspecifically and which complicate further analysis.

Alternatively, antibodies are covalently bound to the matrix. In general, a covalent binding takes place between an amino- and a carboxyl residue or between two SH-residues forming a disulfide bridge. As catalysts, reagents like diflourdinitrobenzol, bromine cyanogen, formaldehyde, glutaraldehyde, hydroxysuccinimide ester and imidate (preferably as dimethyl pimelimidate) are used. For reference see: A one step purification of membrane proteins using a high efficiency immunomatrix. The Journal of Biological Chemistry, Vol. 257, Nr. 18, 1982-09-25, pages 10766-69, and Bioanalytik, F. Lottspeich und H. Zorbas. Spektrum Verlag, Heidelberg, Germany).

As mentioned above, antigen-antibody-complexes can be specifically precipitated by centrifugation, whenever antibodies had been coupled to a matrix. Alternatively, separation of antigen-antibody-complexes can also be achieved with protein-A or protein-G columns. For general information on immunoprecipitations, see: The Journal of Biological Chemistry, Vol. 257, Nr. 18, 1982-09-25, pages 10766-69, and Purification of anti-thyroglobulin IgG from human serum. Clin. Chem. Lab. Med. 2000 Jul. 38 (7), pages 597-602.

The last step of the present invention preferably comprises separation of antigen-antibody-complexes by two-dimensional electrophoresis. Here, proteins are separated in a first dimension according to their isoelectric point within an immobilized pH-gradient, followed by separation in a second dimension according to their molecular weight. For reference see: Görg A, Obermaier C, Boguth G, Harder A, Scheibe B, Wildgruber R, Weiss W. “The current state of two-dimensional electrophoresis with immobilized pH gradients.” Electrophoresis 2000 Apr. 21(6):1037-53.

As used herein, ‘electrophoresis’ refers to the transport of ions within an electric field as a function of form, molecular weight, charge, temperature, viscosity, and field strength. Preferably, gels like polyacrylamide are used as carrier matrices.

Alternatively, proteins are stained with dyes like coomassie blue, silver, Ponceau red or with fluorescent or luminescent reagents after electrophoresis.

It is an objective of the present invention that after separation by electrophoresis, proteins of interest are excised, digested with trypsine and analyzed by mass spectrometry. Alternatively, proteins of interest can be excised, digested with proteases and analyzed be Edman degradation.

Mass spectrometry is conducted e.g. with an matrix-assisted laser distortion ionization/time of flight (MALDI-ToF) spectrometer, an (nanoscale) electrospray ionization (ESI) spectrometer, or quadrupole technology, chemical ionization, or FAB (fast atom bombardment).

Alternatively, proteins can be labeled isotopically with e.g. ³⁵S methionine or ³²P-γATP prior to lysis, or with iodide after lysis. This way, labeled proteins can be visualized by means of autoradiography.

It is an additional objective of the present invention to transfer the proteins to a nitrocellulose- or polyvinylidenfluoride (PVDF)-membrane. The transferred proteins can be visualized by means of staining and are then isolated from the membrane.

Finally, proof of specificity of proteins of interest is preferably achieved using suitable controls like healthy control tissues or the use of serum only (without any tissue=‘serum control’). A serum control allows e.g. the identification of proteins present in sera from cancer patients, only.

Donors for cellular material and autologous/allogeneic sera comprise mammals (especially human beings, primates, rodents like mice, rat, hamster or rabbit) but also infectious organisms like bacteria, parasites, and viruses. As donors of autologous/allogeneic/xenogeneic sera, ascites or pleural fluids can be mammals, especially human beings, primates and rodents like mice, rats, rabbits, and hamster.

The following examples and drawings are provided to further illustrate the present invention and are not to be construed as limiting the scope thereof.

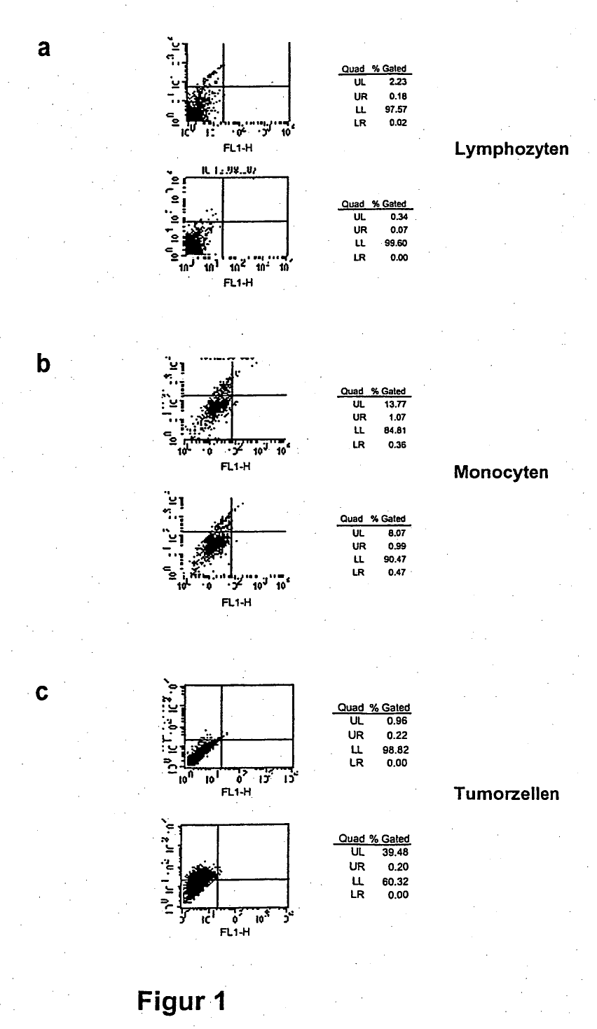

FIG. 1: a flow-cytometric (=FACS) analysis of the humoral immune response in cancer patient GHD-1 after adjuvant treatment with the bispecific antibody BiUII. GHD-1 lymphocytes (A), monocytes (B), and tumor cells (C) were incubated with autologous serum before and after BiUII-therapy. Bound antibodies were detected with an PE-labaled anti-humanIgG3 secondary antibody. BiUII-tharapy induced a significant generation of antibodies that specifically recognize the GHD-1 tumor cell line. Shown is one representative experiment out of three.

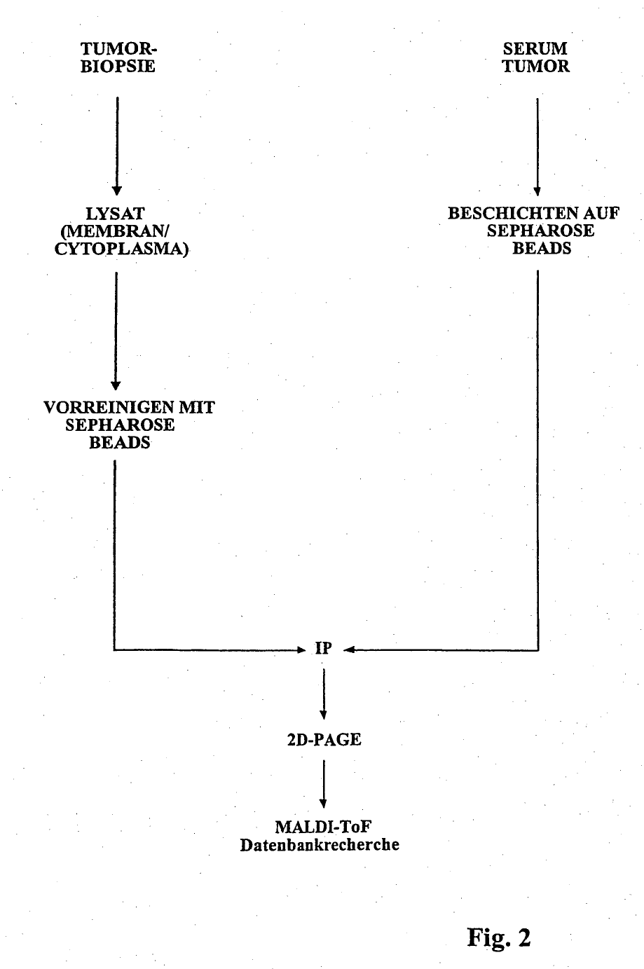

FIG. 2: Flowchart of the AMIDA screening technology.

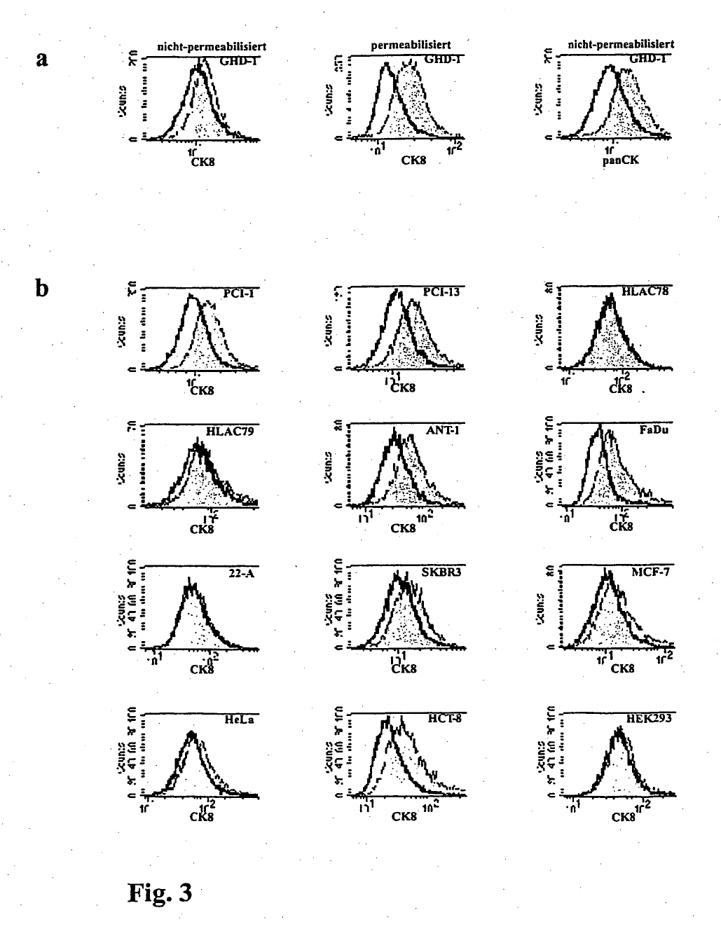

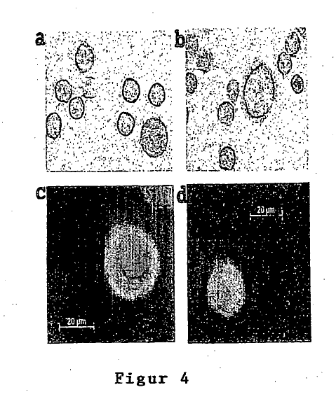

FIG. 3: FACS analysis of the CK8 expression in carcinoma cell lines. A) GHD-1 tumor cells were either left untreated (=non-permeabilized) or were permeabilized and were stained with the CK8-specific antibody 1E8 in combination with a FITC-labeled secondary antibody. B) The non-permeabilized cell lines shown were stained with the CK8-specific antibody 1E8 in combination with a FITC-labeled secondary antibody. Throughout all experiments, dead cell were excluded from analysis by staining with propidiume iodide (except 3A, permeabilized cells). Shown is one representative experiment out of three.

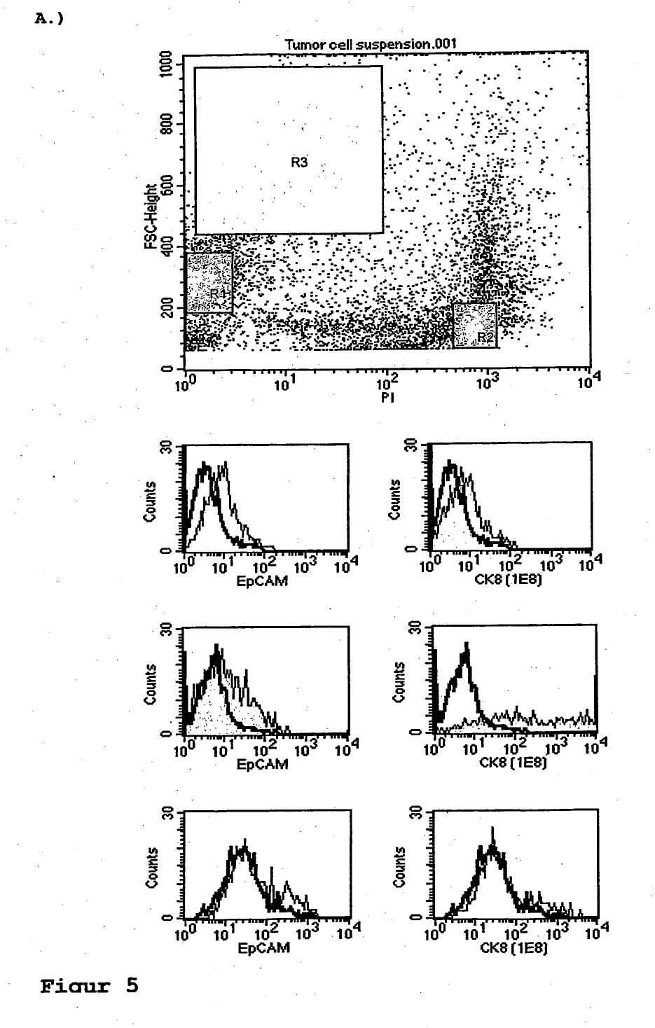

FIG. 4: Immunohistochemistry of CK8 on carcinoma cell lines. A) Cytospins of FaDu- and PCI-1 cells were stained with a CK8-specific antibody in combination with a peroxidase-conjugated secondary antibody. B.) same as A) except that a FITC-labeled secondary antibody was used (green). In addition, DNA was stained with the intercalating dye bis-benzamidine (blue). Shown is one representative experiment out of three.

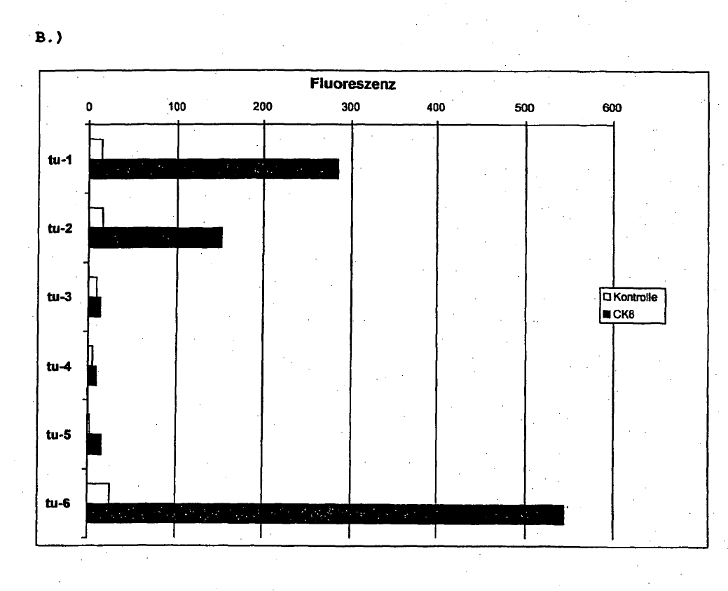

FIG. 5: FACS analysis of the CK8 expression on primary carcinoma cells. A.) single cell suspension generated from biopsies derived from carcinomas of the head and neck were stained with an CK8- or an EpCAM-specific antibody in combination with a FITC-labeled secondary antibody. Cells were gated according to their propidiume iodide (PI) staining (upper histograms). Vital, i.e. PI-negative cells stained positive for EpCAM and CK8 (histograms in the middle). Dead cells (PI-positive) stained negative for EpCAM but expressed high albeit varying amounts of CK8 (lower panels). Shown is a representative result obtained with one out of six tumor biopsies. B.) Shown are the mean CK8 fluorescence intensities and the control fluorescence for six single cell suspensions generated from primary carcinoma.

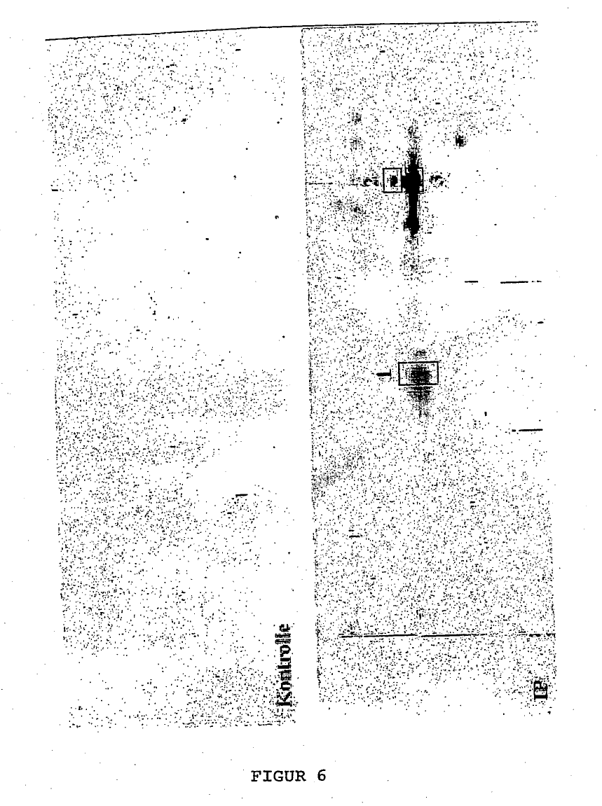

FIG. 6: Two-dimensional gel electrophoresis (2D-PAGE) of antigen-antibody-complexes consisting of Borrelia burgdorferi (B.b.)-antigens and serum antibodies derived from B.b.-infected BALB/c mice. Vital borrelia were lysed in lysisbuffer and unsoluble particles were precipitated by centrifugation. In parallel, BALB/c mice were infected once with B.b. and sera were taken three weeks later. Serum antibodies from infected mice were covalently coupled to sepharose-protein-A beads and used for immuno-precipitation of B.b. lysates. Antigen-antibody-complexes were separated by 2D-PAGE (Amersham IPG strips, pH 3-10L; SDS-PAGE 13%) and visualized by silver-staining.

### EXAMPLES

Cell lines, primary tumor cells, FACS analysis

Cell line GHD-1 was generated from a tumor biopsy derived from a hypopharyngeal carcinoma and was cultivated in standard DMEM medium, supplemented with 10% fetal calf serum (FCS). Single cell suspensions from primary carcinoma-biopsies were generated as follows: biopsies were cut into tiny pieces and incubated for two hours in DMEM containing collagenase (2 mg/ml; type 8, Sigma) and DNAse I (0.2 mg/ml; type IV, Sigma). Cells were washed twice in PBS and resuspended in PBS containing 30/% FCS for immunohistochemistry and FACS analysis. Non-permabilized cells (5×10e5 per sample) were incubated on ice for two hours with the human CK8-specific antibody 1E8 (Huss Diagnostics, Germany), washed with PBS/3% FCS and incubated for one hour on ice with an secondary FITC-labeled antibody. Analysis was performed using a FAC-SCalibur device (BectonDickinson, Germany). Alternatively, cells were fixed for 10 minutes in paraformaldehyde (1%) and permeabilized with Triton (0.2%, 20 min) prior to staining. For all experiments, cells were stained with propidium iodide in order to discriminate vital and dead cells.

Immunoprecipitation, 2D-PAGE, and Mass Spectrometry

Biopsies from primary carcinoma were cut into pieces and homogenized using a 100 μm mesh (Falcon) and cells were washed once in PBS. Cells from primary tumors or GHD-1 cells were incubated for 30 min in hypotonic buffer, containing 10 mM HEPES (pH 7.9), 10 mM KCl, 1.5 mM MgCl₂, 0.1 mM EGTA, and 0.5 mM DTT. Cells were then destroyed mechanically using a 25 G needle and the particulate fraction was centrifuged at 3.000 g and 4° C. 1% Triton and protease inhibitors were then added to the supernatant, which represents the cytoplasmic protein fraction. The pellet, which represents the membrane fraction, was resuspended in 3 volumes of lysis buffer, containing 1% Triton and protease inhibitors. Immunoprecipitation was conducted overnight at 4° C. with sepharose beads, coated with an anti-human IgG3-antibody (only in the case of lysates from GHD-1 cells) and sera from tumor patients. Thereafter, beads were washed in 50 mM Tris, resuspended in 2D-lysis buffer (11M Urea, 4% CHAPS, 10/o DTT, 2.5 mM EDTA, 2.5 mM EGTA) and centrifuged at 42.000 g for 1 hour at 4° C. Proteins in the supernatant were separated in the first dimension by isoelectric focusing in immobilized pH-gradients (pH 3-10 or pH 4-7, IPG-strips, Amersham) and in the second dimension in a 12.5-13% SDS-PAGE according to their molecular weight. Proteins were then visualized by means of coomassie- or silver-staining. Differentially expressed protein spots were excised from the gel, digested with trypsine (2.5 ng/μl; Promega) and analyzed by mass spectrometry (Bruker). Protein search was performed using the Mascot Science database.

Isolation and Identification of Tumor Antigens from Primary Carcinoma Cells using Autologous Sera

The present invention relates to an autologous system, developed for the isolation and identification of antigens, as they are expressed in primary tumor cells where they are targets of a humoral immune response. In order to conduct this method, a single cell suspension was generated from a biopsy derived from a primary hypopharyngeal carcinoma. Cells were then lysed and the proteins were extracted. The lysate was incubated with sepharose protein-A beads for three hours, in order to remove proteins that bind to the beads unspecifically. Sepharose protein-A beads were then coated with autologous serum antibodies and used for immunoprecipitation (IP). Precipitated proteins were separated by two-dimensional electrophoresis according to their isoelectric point and molecular weight, respectively. The following IPs were performed for control purposes: (i) beads coated with serum only, (ii) lysates of autologous non-malignant cells (here: leukocytes), immunprecipitated with serum antibodies, (iii) serum was omitted in order to identified proteins that unspecifically bind to sepharose beads. Protein spots that were exclusively present in IPs of lysates from tumor cells were excised from the gel, digested with trypsine and analyzed by MALDI-ToF mass spectrometry, followed by a Mascot database search for the identification of the proteins. Identified as targets of a humoral immune response were heat shock cognate protein 70 (Hsc70), glucose-regulated protein 78 (Grp78, also known as BiP), and Keratin 9 (CK9).

Treatment of SCCHN-Patients with the Bispecific Antibody BiUII: Characterization of the Induced Humoral Anti-Tumor Response by the Use of AMIDA

Despite improvements in surgical techniques, radiation and chemotherapy, patient with squamous cell carcinoma of the head and neck (SCCHN) still have a poor clinical prognosis. Therefore, adjuvant therapies are being developed, in order to improve the outcome. A promising approach relies on the activation of the immune system in vivo with bispecific antibodies that target tumor cells, T lymphocytes, and Fcγ-positive cells (Lindhofer, H., et al., Preferential species-restricted heavy/light chain pairing in rat/mouse quadromas. Implications for a single-step purification of bispecific antibodies. J Immunol, 1995. 155: pp. 219-25.; Zeidler, R., et al., Simultaneous Activation of T Cells and Accessory Cells by a New Class of Intact Bispecific Antibody Results in Efficient Tumor Cell Killing, Journal of Immunology, 1999. 163: pp. 1246-1252; Zeidler, R., et al., The Fc-region of a new class of intact bispecific antibody mediates activation of accessory cells and NK cells and induces direct phagocytosis of tumour cells. British Journal of Cancer, 2000. 83: pp. 261-266; Zeidler et al., TNF□ contributes to the antitumor activity of a bispecific, trifunctional antibody, Anticancer Research, 2001. 21: pp. 3499-3501). BiUII is such a bispecific antibody that was used for a clinical phase I/II study on patients suffering from hypopharyngeal carcinomas. Patients' sera obtained before and after treatment were collected and tumor biopsies were isolated and cultivated in order to establish continuous cell lines. Induction of a humoral immune response in one patient (GHD-1) was then analyzed by flow cytometry using pre- and post-treatment sera and non-permeabilized autologous tumor cells. Therapy with BiUII induced a strong humoral reaction against the GHD-1 tumor cell line (IgG3 subclass restricted). In contrast, no enhanced binding of antibodies to autologous lymphocytes or monocytes was observed (FIG. 1). Thus, treatment with BiUII induced a tumor-specific humoral immune response.

AMIDA was conducted on patient GHD-1 as follows: pre- and post-treatment sera were separately pre-incubated with sepharose A-αIgG3-beads and then incubated with lysates of protein membrane fractions from GHD-1 cells. Immunoprecipitated proteins were separated by 2D-PAGE and analyzed by mass spectrometry after tryptic digestion. Corresponding proteins were identified using the Mascot Science Protein database. The aims of this experiment were (i) to characterize the humoral immune response, induced by BiUII treatment, and (ii) to conduct AMIDA under stringent and optimal conditions. Nine different spots were identified by AMIDA screening on patient GHD-1. Calculated molecular weights, isoelectric points, and the identity of the corresponding proteins are listed in Table 1. Spot 1 to 4 were identified as human Cytokeratin 8 (probably different modifications thereof), a cytoplasmatic protein expressed in simple epithelia and certain kinds of carcinomas (Southgate, 1999, 13). CK8 is part of a heterodimer (with CK18) that contributes to intermediate filaments and has a molecular weight of 53 kDa and a calculated isoelectric point (pI) of 5.36, corresponding well with results obtained by AMIDA.

Spots 5, 6 and 7 were identified as human immunoglobulin lambda- and kappa-chains, respectively, further indication the induction of a humoral immune response after BiUII therapy. The two remaining spots 8 and 9 were identified as unknown proteins without an entry in any database.

CK8 Cell Surface Expression on Carcinoma Cell Lines and Primary Tumor Cells

In principle, cellular proteins in vital cells can only be recognized by antibodies if they are expressed on the surface. Since CK8 is normally expressed in the cytoplasm only, immunoprecipitation of CK8 from a protein lysate of autologous tumor cells is indicative for an ectopic, surface expression on these cells. We therefore concentrated our investigations onto the characterization of the aberrant surface expression of CK8. To this end, non-permeabilized GHD-1 cells were stained with a CK8-specific antibody in combination with a FITC-labeled secondary antibody. CK8 cell surface expression was measured with a FACSCalibur flow cytometer. A weak albeit consistent CK8 expression was observed on vital non-permeabilized GHD-1 cells. The amount of membrane-associated CK8 represents only a minor fraction as compared to cytoplasmatic CK8 measured in permeablized cells (FIG. 3a). In addition, CK8 expression was determined on a panel of carcinoma cell lines derived from head and neck cancer, cervical cancer, breast cancer and colon cancer, stained with specific antibodies. Nine out of eleven carcinoma cell lines expressed different amounts of surface CK8 (FIG. 3b). The human embryonic kidney cell line 293 (HEK293), which is transformed with oncogenes E1A and E1B from Adenovirus (Graham et al., Characteristics of a human cell line transformed by DNA from human adenovirus type 5.1 General Virology, 1977, Vol 36, pp. 59-74.) was used as a control and stained negative for CK8. Surface staining was not observed in control experiments with antibodies specific to the cytoplasmatic epidermal fatty-acid-binding protein, E-FABP, although E-FABP is very strongly expressed in the cytoplasm (data not shown). In addition, CK8 expression patterns in carcinoma cell lines were determined by immunohistochemistry and immunofluorescence. Cytospins of non-permeabilized FaDu- and PCI-1-cells were stained with a CK8-specific antibody, showing a spotted expression pattern with high local CK8 concentrations at the plasma membrane (FIG. 4). An overlapping expression pattern was observed in double-staining experiments with an antibody specific to the surface pan-carcinoma antigen Ep-CAM, indicative for the membrane-associated localization of CK8 (data not shown).

In order to validate these results in vivo, we next investigated aberrant CK8 surface expression on primary carcinoma cells. To this end, single cell suspensions were prepared from six biopsies of primary tumors of the head and neck and stained with a CK8-specific antibody. Since tumor biopsies also contain non-malignant cells like immune effector cells, tumor cells were distinguished by staining positive for the carcinoma-associated antigen EpCAM. In parallel, vitality of cells was determined by propidium iodide (PI) staining. Both experiments demonstrated that CK8 surface expression is exclusively found on vital cells, which also stain positive for EpCAM (strong EpCAM expression: tu-1, tu-2, tu-6; moderate EpCAM expression: tu-5; weak EpCAM expression: tu-3, tu-4; FIG. 5). In contrast, CK8 surface expression was not detected on EpCAM-negative cells. Dead cells were discriminated by PI-uptake and demonstrated strong and heterogeneous CK8-staining, probably representing cytoplasmatic CK8-staining in dead cells, as also observed in permeabilized cell lines. In summary, these results demonstrate an ectopic CK8 surface expression on squamous cell carcinoma, whereas such an expression is not found on non-malignant cells.

### SUMMARY

Herein, we describe the identification of Hsc70, Grp78, Cytokeratins 8 and 9 and two additional proteins of unknown function as targets for a humoral immune reaction in patients with head and neck cancer with a new method, AMIDA. This immune reaction, provoked by tumor cells within the patients, was used to isolate and characterize tumor-associated proteins my means of AMIDA. CK8 cell surface expression on carcinoma cells was described above extensively. CK8 cell surface expression has also been reported on breast cancer cells where CK8 serves as a membrane-associated receptor for plasminogen and its activator (tissue type plasminogen activator, tPA) (Hembrough, 1996; Cell-surface cytokeratin 8 is the major plasminogen receptor on breast cancer cells and is required for the accelerated activation of cell-associated plasminogen by tissue-type plasminogen activator. J Biol Chem 271 (41), 25684-91 und Hembrough et. al., 1995 A cytokeratin 8-like protein with plasminogen-binding activity is present on the external surfaces of hepatocytes, HepG2 cells and breast carcinoma cell lines. J Cell Sci 108 (3), 1071-82). It is assumed, that CK8 mediates the generation of a ‘protease-system’ at the cell membrane, allowing tumor cells to invade a re-build surrounding tissues. These results correspond with those describing cell surface expression of CK1 and CK8 and breast carcinomas.

More important, these results were validated in primary, freshly isolated tumor cells. Six biopsies derived from hypopharyngeal carcinoma were investigated for CK8 cell surface expression: all stained positive, albeit at various intensities (FIG. 5). Thus, CK8 cell-surface expression on carcinoma cells is neither a cell culture artifact nor are these results due to a fault intrinsic to our detection system. This clear and unique proof rather supports the potential of the present invention. Since carcinoma cell lines of different origin (colon, cervix, breast, head and neck) express CK8 on their cell surface, it is obviously that CK8-specific antibodies are not only present in patients with head and cancer but also in patients with cancer of other organs/localizations. In additional experiments with a cervical carcinoma and a hypopharyngeal carcinoma, we have meanwhile isolated 50 differentially expressed protein spots that are subjects to further investigations.

TABLE 1
Apparent molecular weights and isoelectric points of proteins
isolated from 10 different protein spots, which were reproducibly
identified from AMIDA-screenings on patient GHD-1.

| Spot- | Apparant | Apparant | |
|---|---|---|---|
| No | MW (kDa) | pI (pH) | Protein |
| 1 | 50 | 5.4 | human CK8 |
| 2 | 50 | 5.2 | human CK8 |
| 3 | 50 | 5.1 | human CK8 |
| 4 | 50 | 5.0 | human CK8 |
| 5 | 25 | 5.8 | human Ig-Lambda-chain |
| | | | (precursor) |
| 6 | 20 | 6.2 | human Ig-Kappa-chain |
| 7 | 20 | 6.2 | human Ig-Kappa-chain |
| 8 | 80 | 4.3 | KIAA1273 |
| 9 | 160 | 6.5 | KIAA0373 |

II. Application of AMIDA in a Xenogeneic Setting:
Borrelia burgdorferi and Lyme Disease:

Borrelia burgdorferi (B.b.), which is the pathogen of Lyme disease, is a gram-negative bacteria transferred to human by tick-bites (Ixodes ricinus).

If not or not adequately treated, persistent and chronic arthritis may develop (Lyme arthritis). In addition, chronic infections of the heart and the brain may occur, possibly causing karditis or encephalitis, respectively (Steere, 1997; Steere, 2001). Pathogenesis of lyme disease has been studied in mouse animal models, where a correlation between susceptibility to B.b. and the haplotype of the major histocompatibility complex (MHC) has been demonstrated (Schaible, 1991). An association of certain MHC-haplotypes with chronic lyme disease also exists in human beings.

The Humoral Immune Response in Mice, Infected with B.b.:

The immune response to B.b. and the molecular basis for susceptibility towards B.b. infections are not adequately understood. Activation of CD4+ helper T cells (Th) is essential for the pathogenesis of Lyme arthritis (LA): activation of a Th1 response, characterized by the production of pro-inflammatory cytokines like IFN-gamma, causes induction of LA. In contrast, activation of Th2 cells, and thus the secretion of e.g. IL4, has protective effects against LA (Keane-Myers and Nickell, 1995a; Keane-Myers and Nickell, 1995b; Matyniak et al 1995). Since IL4 is an essential cytokine for B cell activation and maturation, too, a humoral immune response to B.b. infections has been suggested and has been demonstrated experimentally afterward. However, only few B.b. antigens are known in mice, like the membrane proteins OspC and Osp17 (Pohl-Koppe et al., 2001), although protective antibodies are raised (McKisic and Barthold, 2000).

Identification of Target Antigens of the Humoral Immune Response to B.b. by Means of AMIDA

We have investigated the humoral immune response in BALB/c mice before and after infection with B.b. To this end, vital B.b. (3×10e8) were lysed (PBS, 1% Triton-X-100) and unsoluble particles were pelleted by centrifugation. In parallel, BALB/c mice were infected once with 1×10e8 B.b. and sera were take three weeks later. Serum antibodies of infected mice were covalently coupled to sepharose-protein-A beads and used for immunoprecipitation of B.b. lysates (200 μl serum+50 μl sepharose-protein-A). Antigen-antibody-complexes were separated by 2D-PAGE (Amersham IPG strips ph3-10L; SDS-PAGE 113%) und visualized by silver-staining (see FIG. 6). Immunoprecipitated proteins were excised from the gel, digested with trypsine, and analyzed by MALDI-TOF mass spectrometry. Proteins that bound to sepharose-protein-A unspecifically were visualized in a control gel and were excluded from analysis. This way, a B.b.-specific immune response raised against Flagellin (accession number FLLYB3), conserved hypothetical integral membrane protein BB0616 (accession number G70220), and exported protein A (eppA, α-cession number G70176) was demonstrated in infected mice.

III. Application of AMIDA in an Allogeneic Setting:

Identification of Antigens from a Permanently Growing Human Carcinoma Cell Line by Means of Serum Antibodies and AMIDA:

One possible field of application of AMIDA comprises the identification of markers for the detection and diagnosis of diseases. Such a ‘biomarker’ has to fulfill certain prerequisites with respect to frequency and specificity.

This means that the marker in question (e.g. Cytokeratin-8 specific antibodies in patients having a cancer) should be present at a high frequency in patients and at a low frequency in people not having a cancer. In order to address this question, we applied AMIDA in an allogeneic setting, trying to establish a simple and reproducible system. Continuous cell lines (e.g. tumor cell lines) constitute a homogenous and permanently available population and thus represent such a simple system.

To this end, 1×10e7 cells of a continuous human carcinoma cell line (FaDu, ATCC-Nr. HTB-43) were lysed (PBS, 1% Triton-X-100) and unsoluble particles were pelleted by centrifugation. Serum antibodies of a tumor patient were covalently coupled to sepharose-protein-A beads and used for immunoprecipitation of FaDu lysates (200 μl serum+50 μl sepharose-protein-A). Antigen-antibody-complexes were separated by 2D-PAGE (Amersham IPG strips pH 3-10; SDS-PAGE 13%) and visualized by silver-staining (see FIG. 1). Precipitated proteins were excised from the 2D-PAGE, digested with trypsine, and analyzed by MALDI-ToF mass spectrometry. Proteins that bound to sepharose-protein-A unspecifically were visualized in a control gel and were excluded from analysis. This way, we were able to demonstrate the presence of serum antibodies that recognize FaDu-antigens in patients, which can be used for the isolation and identification of allogeneic antigens by means of AMIDA. By Means of AMIDA, the following FaDu-antigens were isolated and identified:

| Protein | Reference |
|---|---|
| Tropomyosin alpha | cytoskeleton-binding protein |
| | (J. Muscle Res Cell Motil 2001; 22: 5-49) |
| BiP (Ig HC-binding protein) | expressed on the surface of rhab- |
| Grp78 (glucose-regulated | domyosarcoma. |
| protein 78) | autoantibodies present in patients |
| | with rheumatic arthritis. |
| Cytokeratin 8 | expressed on the surface of breast |
| | cancer cells (JBC 271: 25684 (1996)) |
| | also identified in an autologous setting |
| | with AMIDA. |

### REFERENCES

Keane-Myers, A. and Nickell, S. P. (1995a) Role of IL-4 and IFN-gamma in modulation of immunity to Borrelia burgdorferi in mice. J Immunol, 155, 2020-2028.
Keane-Myers, A. and Nickell, S. P. (1995b) T cell subset-dependent modulation of immunity to Borrelia burgdorferi in mice. J Immunol, 154, 1770-1776.
Matyniak, I. E., Reiner, S. L., Schaible, U. E., Kramer, M. D., Wallich, R., Tran, T. and Simon, M. M. (1995) T helper phenotype and genetic susceptibility in experimental Lyme disease. J Exp Med, 181, 1251-1254. occurrence of B. burgdorferi-induced arthritis suggest a critical role of T cells in the development of the disease in mice.
McKisic, M. D. and Barthold, S. W. (2000) T-cell-independent responses to Borrelia burgdorferi are critical for protective immunity and resolution of lyme disease. Infect Immun, 68, 5190-5197.
Pohl-Koppe, A., Kaunicnik, A. and Wilske, B. (2001) Characterization of the cellular and humoral immune response to outer surface protein C and outer surface protein 17 in children with early disseminated Lyme borreliosis. Med Microbiol Immunol (Berl), 189, 193-200.
Schaible, U. E., Kramer, M. D., Wallich, R., Tran, T. and Simon, M. M. (1991) Experimental Borrelia burgdorferi infection in inbred mouse strains: antibody response and association of H-2 genes with resistance and susceptibility to development of arthritis. Eur J Immunol, 21, 2397-2405.
Steere, A. C. (1997) Diagnosis and treatment of Lyme arthritis. Med Clin North Am, 81, 179-194.
Steere, A. C. (2001) Lyme disease. N Engl J Med, 345, 115-125.

1. A method for identifying antigens, said method comprising the following steps:
a. Preparing a protein lysate from a doner cellular material to be examined;
b. incubating said protein lysate with allogeneic, xenogeneic or autologous serum, ascites or pleural fluid each containing antibodies, which have developed in the course of a humoral immune response or an autoimmune response against said antigens, to achieve specific binding of said antibodies to said antigens of said protein lysate;
c. separating of the antigen-antibody-complexes
d. detecting of antigens specifically bound by said antibodies

2. A method of claim 1, wherein antibodies directed against immunoglobulins of the donor are additionally added in step b.

3. A method of claim 1, wherein said antigens are tumor antigens, or antigens associated with autoimmune diseases or infections by bacteria, parasites, or viruses.

4. A method of claim 1, wherein said protein lysate is separated into fractions prior to the incubation step.

5. A method of claim 4, wherein said fractionation comprises a separation into a membraneous and a cyctoplasmatic fraction.

6. A method of claim 4, wherein said fractionation comprises a separation into subcellular compartments.

7. A method of claim 4, wherein said fractionation is provided according to the protein size by size exclusion columns.

8. A method of claim 1 wherein said antibodies are coupled to a matrix.

9. A method claim 8 wherein said matrix consists of sepharose, sepharose protein A, sepharose protein G, agarose protein A, or agarose protein G.

10. A method of claim 8 wherein said protein lysate is preincubated with said matrix.

11. A method of claim 1, wherein said antibodies are covalently coupled to the matrix.

12. A method of claim 11, wherein said covalent coupling is provided via amide linkages between amino and carboxyl residues or via disulfide-bridges of two SH residues.

13. A method of one or more of claim 7, wherein said separation of antigen-antibody-complexes is performed by centrifugation or sedimentation.

14. A method of claim 1, wherein said separation of antigen-antibody-complexes is performed by protein A or protein G coated columns.

15. A method of claim 1, wherein said detection of antigens comprises separation of said separated complexes by electrophoresis method.

16. A method of claim 15 wherein two-dimensional electrophoresis is used.

17. A method of claim 15 wherein said antigens are stained with dyes like coomassie blue, silver, Ponceau red, or with fluorecent or luminescent reagents after gel electrophoresis.

18. A method of one claim 1, wherein said cells are labeled with radioactive reagents prior to lyzess???.

19. A method of claim 16, wherein the antigens of interest are excised after electrophoresis separation, digested with a protease, and are analyzed by mass spectrometry.

20. A method of claim 16, wherein said antigens of interest are excised after electrophoresis separation, digested with a protease, and sequenced.

21. A method of claim 16, wherein said antigens are transferred to a nitrocellulose- or polyvinylidenflouride (PVDF)-membrane after electrophoresis and then isolated from this membrane.

22. A method of claim 1, wherein said detection of antigens comprises a comparison of the detected antigens with a control sample especially from healthy cellular material, serum only, or lysate only.

23. A method of claim 1, wherein said donor of said cellular material is a human being, a primate, a rodent or an infectious micro organism.

24. A method of claim 1, wherein said serum, ascites, or pleural fluid is derived human being, a primate, or a rodent.
